(19) United States
(12) Patent Application Publication
(10) Pub. No.: US 2009/0226900 A1
(43) Pub. Date: Sep. 10, 2009

(54) Methods for Reducing Contaminants in Nucleic Acid Sequencing by Synthesis

(75) Inventors: Hazen Babcock, Cambridge, MA (US); Mirna Jarosz, Cambridge, MA (US); Jennifer Colonell, Cambridge, MA (US); Parris Wellman, Reading, MA (US); Geoff Lowman, Cambridge, MA (US); Jayson Bowers, Cambridge, MA (US); John Kepler, Cambridge, MA (US)

Correspondence Address: COOLEY GODWARD KRONISH LLP;ATTN: Patent Group, Suite 1100, 777 - 6th Street, NW, WASHINGTON, DC, 20001, US

(73) Assignee: HELICOS BIOSCIENCES CORPORATION, Cambridge, MA (US)

(21) Appl. No.: 12/043,056

(22) Filed: Mar. 5, 2008

(51) Int. Cl. C12Q 1/68

(52) U.S. Cl. 435/6

(57) ABSTRACT

The disclosure provides methods that improve fidelity of the sequencing-by-synthesis reactions, including methods that reduce impurities and contamination in various reagents, reaction mixtures, and other components of the sequencing systems.

### TECHNICAL FIELD

The invention is in the field of molecular biology and, more specifically, pertains to methods of reducing contaminants in reagents and systems used for nucleic acid synthesis and analysis.

### BACKGROUND OF THE INVENTION

A number of initiatives are currently underway to obtain sequence information directly from millions of individual molecules of DNA or RNA in parallel. Real-time single molecule sequencing-by-synthesis technologies rely on the detection of fluorescent nucleotides as they are incorporated into a nascent strand of DNA that is complementary to the template being sequenced. An example of asynchronous single molecule sequencing by synthesis is illustrated in FIG. 1. As illustrated, oligonucleotides 30-50 bases in length are covalently anchored at the 5′ end to glass cover slips. These anchored strands perform two functions. First, they act as capture sites for the target template strands if the templates are configured with capture tails complementary to the surface-bound oligonucleotides. They also act as primers for the template directed primer extension that forms the basis of the sequence reading. The capture primers function as a fixed position site for sequence determination using multiple cycles of synthesis, detection, and chemical cleavage of the dye-linker to remove the dye. Each cycle consists of adding the polymerase/labeled nucleotide mixture, rinsing, imaging and cleavage of dye. In an alternative method, polymerase is modified with a fluorescent donor molecule and immobilized on a glass slide, while each nucleotide is color-coded with an acceptor fluorescent moiety attached to a gamma-phosphate. The system detects the interaction between a fluorescently-tagged polymerase and a fluorescently modified nucleotide as the nucleotide becomes incorporated into the de novo chain. Other sequencing-by-synthesis technologies also exist.

One important issue in single molecule sequencing is that impurities have a more significant effect on errors and aberrations as compared to traditional bulk sequencing. Single molecule sequencing techniques may be sensitive to incorporation of unlabeled nucleotides, as well as from the incorporation of mismatching nucleotides. For instance, the incorporation of a “dark” nucleotide (i.e., either a natural unmodified dNTP without a functional fluorescent dye or a modified dNTP without a functional fluorescent dye) will produce a false deletion in the sequence read. Even traces of cross-contamination of a dye labeled dNTP into a second dye-labeled dNTP, e.g., dye-dCTP contamination in dye-dATP, give rise to potential substitution errors. Any source of materials (e.g., polymerase, other enzymes, buffers, or labeled nucleotides themselves) might add exogenous enzymes or nucleotides that do not contain a label, thus contributing to the errors. Additionally, impurities may result from the breakdown of dye-labeled nucleotides, which produce nucleotides without a functional label but are capable of outcompeting their labeled analogs.

Defects in sequencing-by-synthesis reactions can arise from: i) defects that cause a particular strand of DNA to stop extending (hereafter referred to as “termination”), ii) defects that cause particular bases to be misread (hereafter referred to as “errors”), and iii) defects that cause loss of the strand from the surface where the DNA was immobilized to the surface (hereafter referred to as “loss”). Some basic examples of the root cause of the errors are: i) termination arising from mismatched base on the 3′-end of the growing primer (e.g., the template is denatured or enzymatically degraded, and the primer no longer functioned as an active substrate for the polymerase); ii) errors arising due to a) impurities in the dye-dNTPs (moieties lacking a functional dye), b) degradation of the dye-dNTPs due to either chemical instability or by action enzymes, and c) selective enzyme action to remove critical functional groups on the dNTPs, e.g., loss of the 5′-phosphate on the tethered nucleobase which prevents multiple base additions in homopolymeric stretches, and iii) loss arising from chemical or enzymatic breaking of attachments to the surface. There are many possible causes of termination, loss and errors, including the contamination of the system with active external agents, such as bacteria, mold or active proteins (e.g., nucleases, proteases and phosphatases) that are introduced to and/or colonize and grow in the system.

Accordingly, there is a need for methods that improve fidelity of the sequencing reactions, particularly, methods that reduce impurities and contamination in various reagents, reaction mixtures, and other components of the sequencing systems.

### SUMMARY OF THE INVENTION

The invention provides methods and compositions for reducing contamination impurities in reagents, reaction mixtures, and other components of sequencing systems. More specifically, the invention provides methods for sequencing a nucleic acid target by synthesis on an automated device which comprises 1) a reaction chamber, 2) one or more storage containers for holding sequencing reagents, and 3) one or more reagent delivery channels for delivery of the sequencing reagents from the storage containers into the reaction chamber.

Automated sequencing by synthesis uses a fluid handling apparatus to introduce reagents to the reaction cell in a cyclic fashion. In use, these fluid handling apparatus can become contaminated and begin to exhibit increasing levels of termination, loss and error. The invention is based, at least in part, on the discovery that periodic cleaning of these systems reduces these effects. In a preferred embodiment, the entire wetted path of the fluidic system is cleaned with a cleaning solution and then rinsed with ultra-pure water after every sequencing run. The cleaning step reduces the error, termination, and/or loss rate(s) of the sequencing (e.g., by at least 10% or more).

Accordingly, in general, methods of the invention include the following steps:

a) conducting one or more cycles of sequencing by synthesis (e.g., single-molecule sequencing on a nucleic acid target on an automated system);

b) cleaning the reagent delivery channels with a cleaning solution (e.g., 100 mM NaOH); and

c) repeating steps a) and b) at least once.

In some embodiments, only a single labeled nucleotide species is added per cycle, for example, as illustrated in FIG. 1 and the cleaning step is performed after every cycle. Optionally, the cleaning step is followed by a rinse in ultra-pure water. In some embodiments, the delivery channel and, optionally, other components of the system are irradiated or replaced to reduce or eliminate bacterial and/or fungal contamination. In some embodiments, the storage containers are pre-treated (chemical treatment, sterilization, etc.) to reduce or eliminate bacterial and/or fungal contamination. In some embodiments, sequencing reagents are treated by heat or irradiation to that end and/or supplemented with a bacteristatic/bactericidal and/or fungistatic/fungicidal agent(s).

### BRIEF DESCRIPTION OF THE FIGURES

FIGS. 1A and 1B illustrate a typical process of single molecule sequencing by synthesis. 1) “Capture probes” (T(50) oligonucleotides also functioning as primers) are covalently bound with “5′ down” to a surface. 2) Genomic DNA is fragmented, and a polyA tail and a Cy3 label are added at 3′ of each fragment. These DNA templates are then hybridized to the capture probes. 3) The captured templates are imaged to establish their location. 4) The captured templates are incubated with a Cy5-labeled nucleotide and a polymerase mixture to allow the polymerization reaction to proceed. 5) The surface is rinsed to wash out unincorporated nucleotides and other reagents. 6) The incorporated nucleotides are imaged and associated with each template by their location. 7) The Cy5 label is chemically cleaved off. 8) The process is repeated with another type of nucleotide.

### DETAILED DESCRIPTION OF THE INVENTION

The invention provides methods and compositions for reducing impurities in reagents, reaction mixtures, and other components of the sequencing system.

One method for dealing with contamination is to use reagents that are appropriately filtered and treated to prevent growth. Exemplary methods includes: gamma-irradiation, e-beam irradiation, high intensity light, high intensity ultraviolet light, ultra-filtration at point of fill, high temperature (>90° C.) exposure of reagents for fixed periods of time and introduction of bactericidal, fungicidal or bacteristatic and fungistatic reagents such as sodium azide, triclosan, aldehydes, metal oxide nanoparticles, bleach, detergents and a wide range of antibiotic and chemotherapeutic reagents.

It was also found that pre-cleaning and pre-treating the storage containers reduces the bacterial and fungal contamination to unmeasurable levels. We have employed detergents and alcohols in this method. In a preferred embodiment, all storage containers are pre-washed with a 70% ethanol/30% water solution and final rinsed with “ultra-pure” water that is purified by reverse osmosis. Other reagents include bleach and sodium hydroxide. Other methods include molding the containers in sterile conditions, and irradiating or heat treating the storage containers before filling.

Cyclic sequencing by synthesis uses a fluid-handling apparatus to introduce reagents to the reaction cell in a cyclic fashion. In use, such a fluid-handling apparatus can become contaminated and begin to exhibit increasing levels of termination, loss and error. The invention is based, in part, on the discovery that periodic cleaning of these systems is essential to reducing these effects. In a preferred embodiment, the entire wetted path of the fluidic system is cleaned with an active reagent and then rinsed with ultra-pure water after every sequencing run. In a preferred embodiment, the active reagent is 100 mM NaOH dissolved in ultra-pure water. In other embodiments, a different set of reagents is used on a periodic basis to effect a deep clean.

In a further embodiment, the rinse volumes and soak times are run automatically by the machine. In yet another embodiment, the entire wetted path of the system or such portions as are necessary to reduce termination, loss and errors to acceptable levels is made disposable and is replaced before every sequencing run. In a further embodiment, the entire wetted fluid path is irradiated with X-ray, e-beam or high intensity light between uses.

Accordingly, the invention provides methods of sequencing a nucleic acid target by synthesis. The methods including the following steps:

a) conducting one or more cycles of sequencing by synthesis on a nucleic acid target on an automated device comprising 1) a reaction chamber, 2) one or more storage containers for holding sequencing reagents, and 3) one or more reagent delivery channels for delivery of the sequencing reagents from the storage containers into the reaction chamber;

b) cleaning the reagent delivery channels with a first cleaning solution; and

c) repeating steps a) and b) at least once;

thereby to reduce the error, termination, and/or loss rate(s) of the sequencing (e.g., by at least 10%, 15%, 20%, 25%, 50% or more as a result of step b). In some embodiments, step b) is performed after every cycle in step a)).

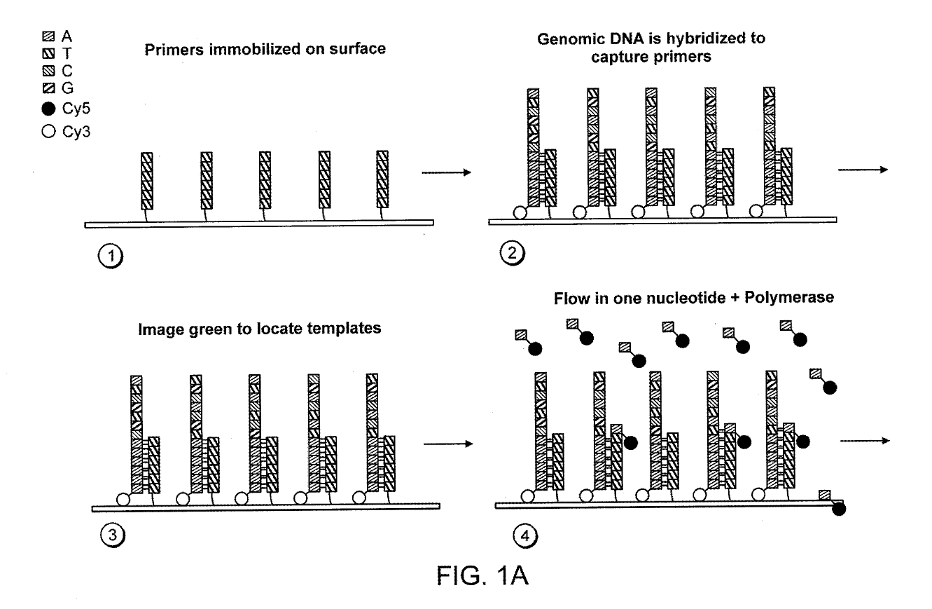

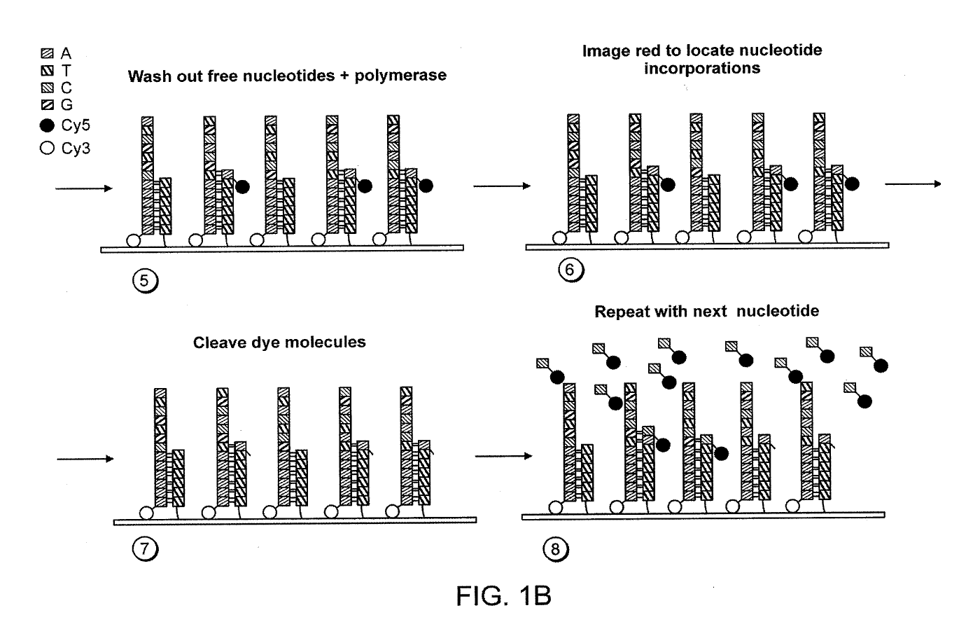

In some embodiments, the sequencing is performed at a single molecule resolution. In some embodiments, as illustrated in FIG. 1, only a single labeled nucleotide species is added per cycle.

In some embodiments, the methods of the invention further include cleaning the reagent delivery channels with a second cleaning solution different from the first cleaning solution. The first and/or the second cleaning solutions can be a solution that has a pH of 9, 9.5, 10, 10.5, or higher. For example, the cleaning solution may contain a strong base, e.g., from 1 mM to 1 M NaOH or KOH, e.g., 100 mM NaOH.

In some embodiments, the methods of the invention further include rinsing the delivery channels with ultra-pure water.

In some embodiments, the delivery channels are disposable and are replaced following a number of cycles (e.g., every 100, 500, 1000, 10000, 100000, or more cycles). Alternatively, the delivery channels are irradiated following a number of cycles, for example, with X-ray, e-beam, high intensity visible light, and/or ultraviolet light.

In some embodiments, the storage containers are pre-treated with a solution reducing bacterial or fungal contamination. For example, storage containers may be pre-treated by rinsing in 70%/30% ethanol/water, followed by a rinse in ultra-pure water. Alternatively, storage containers may be pre-treated with a solution of a) sodium hypochlorite, b) sodium hydroxide, and/or c) detergent, followed by a rinse in ultra-pure water. Storage containers may also be pre-treated by sterilization by various means including high temperature, X-ray, e-beam, high intensity visible light, and/or ultraviolet light.

In some embodiments, one or more sequencing reagents are pre-heated to a temperature of 90° C. or higher for a fixed period of time. In addition, or alternatively, the reagents may be supplemented with a bacteristatic/bactericidal and/or fungistatic/fungicidal agent such as, for example, sodium azide, triclosan, aldehyde, metal oxide nanoparticles, sodium hypochlorite, a detergent, an antibiotic, and a chemotherapeutic agent.

### Sequencing Platforms

The invention can be used on any suitable sequencing-by-synthesis platform as well as on any suitable sequencing-by-hybridization platform. As described above, four major sequencing-by-synthesis platforms are currently available: the Genome Sequencers from Roche/454 Life Sciences, the 1G Analyzer from Illumina/Solexa, the SOLiD system from Applied BioSystems, and the Heliscope system from Helicos Biosciences. Sequencing-by-synthesis platforms have also been described by Pacific BioSciences and VisiGen Biotechnologies. Sequencing-by-hybridization platforms include, for example, those by Affymetrix and Complete Genomics Each of these platforms can be used in the methods of the invention. In some embodiments, the sequencing platforms used in the methods of the present invention have one or more of the following features:

1) four differently optically labeled nucleotides are utilized (e.g., 1G Analyzer, Pacific BioSciences, and Visigen);

2) sequencing-by-ligation is utilized (e.g., SOLiD);

3) pyrophosphate detection is utilized (e.g., Roche/454);

4) four identically optically labeled nucleotides are utilized (e.g., Helicos);

5) fluorescent energy transfer (FRET) is utilized (e.g., Visigen).

In some embodiments, a plurality of nucleic acid molecules being sequenced is bound to a support. To immobilize the nucleic acid on a support, a capture sequence/universal priming site can be added at the 3′ and/or 5′ end of the template. The nucleic acids may be bound to the solid support by hybridizing the capture sequence to a complementary sequence covalently attached to the solid support. The capture sequence (also referred to as a universal capture sequence) is a nucleic acid sequence complementary to a sequence attached to a solid support that may dually serve as a universal primer. In some embodiments, the capture sequence is polyNₙ, wherein N is U, A, T, G, or C, n≧5, e.g., 20-70, 40-60, e.g., about 50. For example, the capture sequence could be polyT₄₀₋₅₀ or its complement.

As an alternative to a capture sequence, a member of a coupling pair (such as, e.g., antibody/antigen, receptor/ligand, or the avidin-biotin pair as described in, e.g., US Patent Application No. 2006/0252077) may be linked to each fragment to be captured on a surface coated with a respective second member of that coupling pair.

The solid support may be, for example, a glass surface such as described in, e.g., US Patent App. Pub. No. 2007/0070349. The surface may be coated with an epoxide, polyelectrolyte multilayer, or other coating suitable to bind nucleic acids. In preferred embodiments, the surface is coated with epoxide and a complement of the capture sequence is attached via an amine linkage. The surface may be derivatized with avidin or streptavidin, which can be used to attach to a biotin-bearing target nucleic acid. Alternatively, other coupling pairs, such as antigen/antibody or receptor/ligand pairs, may be used. The surface may be passivated in order to reduce background. Passivation of the epoxide surface can be accomplished by exposing the surface to a molecule that attaches to the open epoxide ring, e.g., amines, phosphates, and detergents.

Subsequent to the capture, the sequence may be analyzed, for example, by single molecule detection/sequencing, e.g., as described in U.S. Pat. No. 7,283,337, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or can be done in a step-and-repeat mode. For real-time analysis, different optical labels to each nucleotide may be incorporated and multiple lasers may be utilized for stimulation of incorporated nucleotides.

### Target Nucleic Acids

The length of the target nucleic acid may vary. The average length of the target nucleic acid may be, for example, at least 300, 350, 400, 450, 500, 550, 600, 700, 800, 900, 1000, 2000, 3000, 4000, 5000 nts or longer. In some embodiments, the length of the target is between 300 and 5000 nts, 400 and 4000 nts, or 500 and 3000 nts.

Target nucleic acids can come from a variety of sources. For example, nucleic acids can be naturally occurring DNA or RNA (e.g., mRNA or non-coding RNA) isolated from any source, recombinant molecules, cDNA, or synthetic analogs. For example, the target nucleic acid may include whole genes, gene fragments, exons, introns, regulatory elements (such as promoters, enhancers, initiation and termination regions, expression regulatory factors, expression controls, and other control regions), DNA comprising one or more single-nucleotide polymorphisms (SNPs), allelic variants, and other mutations. The target nucleic acid may also be tRNA, rRNA, ribozymes, splice variants, or antisense RNA.

Target nucleic acids may be obtained from whole organisms, organs, tissues, or cells from different stages of development, differentiation, or disease state, and from different species (human and non-human, including bacteria and virus). Various methods for extraction of nucleic acids from biological samples are known (see, e.g., Nucleic Acids Isolation Methods, Bowein (ed.), American Scientific Publishers, 2002). Typically, genomic DNA is obtained from nuclear extracts that are subjected to mechanical shearing to generate random long fragments. For example, genomic DNA may be extracted from tissue or cells using a Qiagen DNeasy Blood & Tissue Kit following the manufacturer's protocols.

Other details and variations of the sequencing methods are provided below.

### Other General Considerations

A. Nucleotides—Nucleotides useful in the invention include any nucleotide or nucleotide analog, whether naturally occurring or synthetic. For example, preferred nucleotides include phosphate esters of deoxyadenosine, deoxycytidine, deoxyguanosine, deoxythymidine, adenosine, cytidine, guanosine, and uridine. Other nucleotides useful in the invention comprise an adenine, cytosine, guanine, thymine base, a xanthine or hypoxanthine; 5-bromouracil, 2-aminopurine, deoxyinosine, or methylated cytosine, such as 5-methylcytosine, and N4-methoxydeoxycytosine. Also included are bases of polynucleotide mimetics, such as methylated nucleic acids, e.g., 2′-O-methRNA, peptide nucleic acids, modified peptide nucleic acids, locked nucleic acids and any other structural moiety that can act substantially like a nucleotide or base, for example, by exhibiting base-complementarity with one or more bases that occur in DNA or RNA and/or being capable of base-complementary incorporation, including chain-terminating analogs.

Nucleotides for nucleic acid sequencing, according to the invention, preferably comprise a detectable label that is directly or indirectly detectable. Preferred labels include optically detectable labels, such as fluorescent labels. Examples of fluorescent labels include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-d iethylamino-3-(4′-isothiocyanatophenyl)-4-methylcoumarin; diethylenetriamine pentaacetate; 4,4′-diisothiocyanatodihydrostilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron® Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. Preferred fluorescent labels are cyanine-3 and cyanine-5. Additional fluorescent dyes that can be used in the methods of the invention include ATTO dyes (such as, e.g., ATTO 390, 425, 465, 488, 495, 520, 532, 550, 565, 590, 594, 610, 611X, 620, 633, 635, 637, 647, 647N, 655, 680, 700, 725, and 740) available from Atto Technologies (Germany).

Labels other than fluorescent labels are contemplated, including other optically detectable labels. In some embodiments, a labeled nucleotide comprises a fluorescent label attached to the nitrogenous base, optionally, via a disulfide, such as illustrated by Formula I and Formula II below.

B Nucleic Acid Polymerases—Nucleic acid polymerases generally useful in the invention include DNA polymerases, RNA polymerases, reverse transcriptases, and mutant or altered forms of any of the foregoing. DNA polymerases and their properties are described in detail in, among other places, DNA Replication 2nd edition, Komberg and Baker, W. H. Freeman, New York, N.Y. (1991). Known conventional DNA polymerases useful in the invention include, but are not limited to, Pyrococcus furiosus (Pfu) DNA polymerase (Lundberg et al. (1991) Gene, 108:1, Stratagene), Pyrococcus woesei (Pwo) DNA polymerase (Hinnisdaels et al. (1996), Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh et al. (1977) Biochim. Biophys. Acta, 475:32), Thermococcus litoralis (Tli) DNA polymerase (also referred to as Vent® DNA polymerase, Cariello et al. (1991) Polynucleotides Res., 19:4193; New England Biolabs), 9° Nm® DNA polymerase (New England Biolabs), Stoffel fragment, ThermoSequenase® (Amersham Pharmacia Biotech UK), Therminator® (New England Biolabs), Thermotoga maritima (Tma) DNA polymerase (Diaz et al. (1998) Braz. J. Med. Res., 31:1239), Thermus aquaticus (Taq) DNA polymerase (Chien et al. (1976) J. Bacteoriol., 127:1550), DNA polymerase, Pyrococcus kodakaraensis KOD DNA polymerase (Takagi et al. (1997) Appl. Environ. Microbiol., 63:4504), JDF-3 DNA polymerase (from thermococcus sp. JDF-3, PCT Patent Application Publication WO 01/32887), Pyrococcus GB-D (PGB-D) DNA polymerase (also referred as Deep Vent® DNA polymerase, Juncosa-Ginesta et al. (1994) Biotechniques, 16:820; New England Biolabs), UlTma DNA polymerase (from thermophile Thermotoga maritima; Diaz et al. (1998) Braz. J. Med. Res., 31:1239; PE Applied Biosystems), Tgo DNA polymerase (from thermococcus gorgonarius, Roche Molecular Biochemicals), E. coli DNA polymerase I (Lecomte et al. (1983) Polynucleotides Res., 11:7505), T7 DNA polymerase (Nordstrom et al. (1981) J. Biol. Chem., 256:3112), and archaeal DP11/DP2 DNA polymerase II (Cann et al. (1998) Proc. Natl. Acad. Sci. USA, 95:14250-5).

While thermophilic polymerases are contemplated by the invention, preferred polymerases are mesophilic. Mesophilic DNA polymerases include, but are not limited to, E. coli DNA polymerase I and Klenow (exo⁻) fragment. Polymerases, irrespective of source, are preferably exonuclease-deficient in many implementations.

Reverse transcriptases useful in the invention include, but are not limited to, reverse transcriptases from HIV, HTLV-1, HTLV-II, FeLV, FIV, SIV, AMV, MMTV, MoMuLV and other retroviruses (see Levin (1997) Cell, 88:5-8; Verma (1977) Biochim. Biophys. Acta, 473:1-38; Wu et al. (1975) CRC Crit. Rev. Biochem., 3:289-347).

C. Surfaces/Solid support—In a preferred embodiment, nucleic acid template molecules are attached to a solid support (“substrate”). Substrates for use in the invention can be two- or three-dimensional and can comprise a planar surface (e.g., a glass slide) or can be shaped. A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites.

Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a nucleic acid. Substrates can include planar arrays or matrices capable of having regions that include populations of template nucleic acids or primers. Examples include nucleoside-derivatized CPG and polystyrene slides; derivatized magnetic slides; polystyrene grafted with polyethylene glycol, and the like.

In one embodiment, a substrate is coated to allow optimum optical processing and nucleic acid attachment. Substrates for use in the invention can also be treated to reduce background. Exemplary coatings include epoxides, and derivatized epoxides (e.g., with a binding molecule, such as streptavidin). The surface can also be treated to improve the positioning of attached nucleic acids (e.g., nucleic acid template molecules, primers, or template molecule/primer duplexes) for analysis. As such, a surface according to the invention can be treated with one or more charge layers (e.g., a negative charge) to repel a charged molecule (e.g., a negatively charged labeled nucleotide). For example, a substrate according to the invention can be treated with polyallylamine followed by polyacrylic acid to form a polyelectrolyte multilayer. The carboxyl groups of the polyacrylic acid layer are negatively charged and thus repel negatively charged labeled nucleotides, improving the positioning of the label for detection. Coatings or films applied to the substrate should be able to withstand subsequent treatment steps (e.g., photoexposure, boiling, baking, soaking in warm detergent-containing liquids, and the like) without substantial degradation or disassociation from the substrate.

Examples of substrate coatings include, vapor phase coatings of 3-aminopropyltrimethoxysilane, as applied to glass slide products, for example, from Erie Glass (Portsmouth, N.H.). In addition, generally, hydrophobic substrate coatings and films aid in the uniform distribution of hydrophilic molecules on the substrate surfaces. Importantly, in those embodiments of the invention that employ substrate coatings or films, the coatings or films that are substantially non-interfering with primer extension and detection steps are preferred. Additionally, it is preferable that any coatings or films applied to the substrates either increase template molecule binding to the substrate or, at least, do not substantially impair template binding.

Various methods can be used to anchor or immobilize the primer to the surface of the substrate. The immobilization can be achieved through direct or indirect bonding to the surface. The bonding can be by covalent linkage. See, Joos et al. (1997) Analytical Biochemistry, 247:96-101; Oroskar et al. (1996) Clin. Chem., 42:1547-1555; and Khandjian (1986) Mol. Bio. Rep., 11:107-11. A preferred attachment is direct amine bonding of a terminal nucleotide of the template or the primer to an epoxide integrated on the surface. The bonding also can be through non-covalent linkage. For example, biotin-streptavidin (Taylor et al. (1991) J. Phys. D: Appl. Phys., 24:1443) and digoxigenin with anti-digoxigenin (Smith et al. (1992) Science, 253:11220) are common tools for anchoring nucleic acids to surfaces and parallels. Alternatively, the attachment can be achieved by anchoring a hydrophobic chain into a lipid monolayer or bilayer. Other methods known in the art for attaching nucleic acid molecules to substrates can also be used.

D. Detection—Any detection method may be used that is suitable for the type of label employed. Thus, exemplary detection methods include radioactive detection, optical absorbance detection, e.g., UV-visible absorbance detection, optical emission detection, e.g., fluorescence or chemiluminescence. For example, extended primers can be detected on a substrate by scanning all or portions of each substrate simultaneously or serially, depending on the scanning method used. For fluorescence labeling, selected regions on a substrate may be serially scanned one-by-one or row-by-row using a fluorescence microscope apparatus, such as described in Fodor (U.S. Pat. No. 5,445,934) and Mathies et al. (U.S. Pat. No. 5,091,652). Devices capable of sensing fluorescence from a single molecule include the scanning tunneling microscope (siM) and the atomic force microscope (AFM). Hybridization patterns may also be scanned using a CCD camera (e.g., Model TE/CCD512SF, Princeton Instruments, Trenton, N.J.) with suitable optics (Ploem, in Fluorescent and Luminescent Probes for Biological Activity, Mason (ed.), Academic Press, Landon, pp. 1-11 (1993), such as described in Yershov et al. (1996) Proc. Natl. Acad. Sci., 93:4913, or may be imaged by TV monitoring. For radioactive signals, a PhosphorImager™ device can be used (Johnston et al. (1990) Electrophoresis, 13:566; Drmanac et al. (1992) Electrophoresis, 13:566). Other commercial suppliers of imaging instruments include General Scanning Inc., (Watertown, Mass.; genscan.com), Genix Technologies (Waterloo, Ontario, Canada; confocal.com), and Applied Precision Inc. Such detection methods are particularly useful to achieve simultaneous scanning of multiple attached template nucleic acids.

A number of approaches can be used to detect incorporation of fluorescently-labeled nucleotides into a single nucleic acid molecule. Optical setups include near-field scanning microscopy, far-field confocal microscopy, wide-field epi-illumination, light scattering, dark field microscopy, photoconversion, single and/or multiphoton excitation, spectral wavelength discrimination, fluorophore identification, evanescent wave illumination, and total internal reflection fluorescence (TIRF) microscopy. In general, certain methods involve detection of laser-activated fluorescence using a microscope equipped with a camera. Suitable photon detection systems include, but are not limited to, photodiodes and intensified CCD cameras. For example, an intensified charge couple device (ICCD) camera can be used. The use of an ICCD camera to image individual fluorescent dye molecules in a fluid near a surface provides numerous advantages. For example, with an ICCD optical setup, it is possible to acquire a sequence of images (“movies”) of fluorophores.

Some embodiments of the present invention use TIRF microscopy for two-dimensional imaging. TIRF microscopy uses totally internally reflected excitation light and is well known in the art. See, e.g., nikon-instruments.jp/eng/page/products/tirf.aspx. In certain embodiments, detection is carried out using evanescent wave illumination and total internal reflection fluorescence microscopy. An evanescent light field can be set up at the surface, for example, to image fluorescently labeled nucleic acid molecules. When a laser beam is totally reflected at the interface between a liquid and a solid substrate (e.g., glass), the excitation light beam penetrates only a short distance into the liquid. The optical field does not end abruptly at the reflective interface, but its intensity falls off exponentially with distance. This surface electromagnetic field, called the “evanescent wave,” can selectively excite fluorescent molecules in the liquid near the interface. The thin evanescent optical field at the interface provides low background and facilitates the detection of single molecules with high signal-to-noise ratio at visible wavelengths. The evanescent field also can image fluorescently-labeled nucleotides upon their incorporation into the attached template/primer complex in the presence of a polymerase. Total internal reflectance fluorescence microscopy is then used to visualize the attached template/primer duplex and/or the incorporated nucleotides with single molecule resolution.

All publications, patents, patent applications, and biological sequences cited in this disclosure are incorporated by reference in their entirety.

1. A method of sequencing a nucleic acid target by synthesis, the method comprising:
a) conducting one or more cycles of sequencing by synthesis on a nucleic acid target on an automated device comprising a reaction chamber, one or more storage containers for holding sequencing reagents, and one or more reagent delivery channels for delivery of the sequencing reagents from the storage containers into the reaction chamber;
b) cleaning the reagent delivery channels with a first cleaning solution; and
c) repeating steps a) and b) at least once;
thereby to reduce the error, termination, and/or loss rate(s) of the sequencing.

2. The method of claim 1, wherein the error, termination, or loss rate is reduced by at least 10% as a result of step b).

3. The method of claim 1, wherein the target nucleic acid is immobilized on a support.

4. The method of claim 1, wherein the sequencing is observed at a single molecule resolution.

5. The method of claim 1, wherein only a single labeled nucleotide species is added per cycle.

6. The method of claim 1, wherein the first cleaning solution has a pH of 9 or higher.

7. The method of claim 1, wherein the first cleaning solution contains approximately 100 mM NaOH.

8. The method of claim 1, further comprising cleaning the reagent delivery channels with a second cleaning solution different from the first cleaning solution.

9. The method of claim 1, further comprising rinsing the delivery channels, and optionally the reaction chamber, with ultra-pure water.

10. The method of claim 1, wherein step b) is performed after every cycle in step a).

11. The method of claim 1, wherein the delivery channels are disposable and are replaced following a number of cycles.

12. The method of claim 1, wherein the delivery channels are irradiated following a number of cycles.

13. The method of claim 12, wherein the delivery channels are irradiated with X-ray, e-beam, high intensity visible light, and/or ultraviolet light.

14. The method of claim 1, further comprising pre-treating storage containers with a solution reducing bacterial or fungal contamination.

15. The method of claim 14, wherein the storage containers are pre-treated by rinsing in 70%/30% ethanol/water, followed by a rinse in ultra-pure water.

16. The method of claim 14, wherein the storage containers are pre-treated with a solution of a) sodium hypochlorite, b) sodium hydroxide, and/or c) detergent, followed by a rinse in ultra-pure water.

17. The method of claim 14, wherein the storage containers are pre-treated by sterilization.

18. The method of claim 1, wherein one or more sequencing reagents are pre-heated to a temperature of 90° C. or higher for a fixed period of time.

19. The method of claim 1, wherein one or more sequencing reagents are supplemented with a bacteristatic/bactericidal and/or fungistatic/fungicidal agent.

20. The method of claim 1, wherein the bacteristatic/bactericidal and/or fungistatic/fungicidal agent is/are chosen from sodium azide, triclosan, aldehyde, metal oxide nanoparticles, sodium hypochlorite, a detergent, an antibiotic, and a chemotherapeutic agent.
